(19) United States
(12) Patent Application Publication
(10) Pub. No.: US 2023/0395222 A1
(43) Pub. Date: Dec. 7, 2023

(54) SYSTEM, ELECTRONIC DEVICE, METHOD, AND PROGRAM FOR ADJUSTING INTAKE OF DRUG

(71) Applicant: CureApp, Inc., Tokyo (JP)

(72) Inventors: Kohta Satake, Chuo-ku, Tokyo (JP); Masaki Aijima, Chuo-ku, Tokyo (JP)

(21) Appl. No.: 18/032,487

(22) PCT Filed: Jun. 30, 2021
(86) PCT No.: PCT/JP2021/024751

§ 371 Date: Apr. 18, 2023

(30) Foreign Application Priority Data
Oct. 22, 2020 (JP) 2020-177265

(51) Int. Cl. G16H 20/10; G16H 50/30; G16H 10/40

(52) U.S. Cl. CPC G16H 20/10; G16H 50/30; G16H 10/40

(57) ABSTRACT

A system that adjusts an intake target of a drug indicating an amount of the drug to be taken by a person to be medicated, the system configured to: acquire intake information of a drug taken by a person to be medicated based on an intake target of the drug; determine, based on the intake information, an occurrable timing at which a side effect of the drug taken is likely to occur; present inquiry information that inquires whether a side effect has occurred in the person to be medicated at the occurrable timing; acquire response information that is input in response to the inquiry and indicates whether a side effect has occurred; change the intake target of the drug based on the response information; and present the intake target changed.

### TECHNICAL FIELD

This disclosure relates to a system, an electronic device, a method, and a program for adjusting intake of a drug.

### BACKGROUND

In conventional medical care, a dosage of a drug is determined at the time of a face-to-face medical consultation with a medical practitioner. With the recent development of information communication technologies, a system for managing a patient for whom a drug is prescribed such that the patient can take, by using an information terminal such as a smartphone possessed by the patient, a prescribed drug in a prescribed amount at prescribed time determined by a medical practitioner has been developed (JP 2018-147463 A).

Efficacy and side effects depending on a dosage of a drug vary among individuals. For this reason, to get a better effect of a drug, a drug sometimes needs to be administered more than a generally prescribed dosage and administration. However, in conventional medical practice, a medical practitioner has difficulty in detecting side effects in a patient (person to be medicated) in a more timely manner, and thus a conservative dosage is set as a standard, and only such a moderate dosage as a standard determined in that manner can be applied, failing to get a sufficient effect of a drug in some instances. Similarly, drugs to be sold with no instructions from a medical practitioner are taken by a person to be medicated in a moderate intake as a predetermined amount determined at the time of the production or the like, failing to get a sufficient effect of a drug in some instances.

### SUMMARY

We provide a system for adjusting an intake target of a drug indicating an amount of the drug to be taken by a person to be medicated. The system acquires intake information of a drug taken by a person to be medicated based on an intake target of the drug, determines, based on the intake information, an occurrable timing at which a side effect caused by the drug taken is likely to occur, presents inquiry information for inquiring whether a side effect has occurred in the person to be medicated at the occurrable timing, acquires response information that is input in response to the inquiry and indicates whether a side effect has occurred, changes the intake target of the drug based on the response information, and presents the intake target changed.

The occurrable timing may be an occurrable period during which a side effect is likely to occur, and presenting the inquiry information may include presenting the inquiry information in response to a presentation request of a user during the occurrable period.

The occurrable timing at which the side effect is likely to occur may be determined based on an occurrable timing determinant, and the system may further correct an occurrable timing determinant based on the response information.

The occurrable timing determinant may be an estimated time to a maximum plasma concentration.

The system may determine a probability of occurrence of a side effect at a timing to present inquiry information, and presenting the inquiry may be preferentially presenting a side effect in which the high probability of occurrence determined is high.

The system may determine a probability of occurrence of a side effect at a timing to present inquiry information, and presenting the inquiry may be presenting the probability of occurrence determined along with a side effect.

The system may repeatedly perform acquisition of the intake information of the drug, determination of the occurrable timing, presentation of the inquiry information, acquisition of the response information, change of the intake target of the drug, and presentation of the intake target changed, and the change of the intake target of the drug may include increasing the intake target of the drug when the response information indicates that no side effect occurs, and decreasing the intake target of the drug when the response information indicates that a side effect has occurred.

The system may repeatedly perform acquisition of the intake information of the drug, determination of the occurrable timing, presentation of the inquiry information, acquisition of the response information, change of the intake target of the drug, and presentation of the intake target changed, and the intake target of the drug may be decreased after the response information indicates that a side effect has occurred, and the intake target of the drug may be maintained after the response information indicates that no side effect occurs.

The inquiry information may include information for inquiring whether efficacy of the drug has occurred in a person to be medicated, the response information may include information indicating whether efficacy of the drug has occurred, and the change of the intake target of the drug may be performed based on response information including the information indicating whether the efficacy of the drug has occurred.

The system may repeatedly perform acquisition of the intake information of the drug, determination of the occurrable timing, presentation of the inquiry information, acquisition of the response information, change of the intake target of the drug, and presentation of the intake target changed, and the intake target of the drug may be decreased when the response information indicates that a side effect has occurred, and the intake target of the drug may be increased when the response information indicates that no side effect occurs and that no efficacy of the drug occurs.

The system may repeatedly perform acquisition of the intake information of the drug, determination of the occurrable timing, presentation of the inquiry information, acquisition of the response information, change of the intake target of the drug, and presentation of the intake target changed and include determining that the response information indicates that no side effect occurs may be included when the response information indicates that no side effect occurs a predetermined number of times or more.

An electronic device adjusts an intake target of a drug indicating an amount of the drug to be taken by a person to be medicated The electronic device is configured to receive intake information of a drug taken by a person to be medicated, determine, based on the intake information, an occurrable timing at which a side effect caused by the drug taken is likely to occur, transmit information for presenting inquiry information for inquiring whether a side effect has occurred in the person to be medicated at the occurrable timing, receive response information that is input in response to the inquiry and indicates whether a side effect has occurred, change the intake target of the drug based on the response information, and present the intake target changed.

The electronic device may adjust an intake target indicating an amount and indicates of a drug to be taken by a person to be medicated The electronic device is configured to transmit intake information of a drug to be taken by a person to be medicated input, present an inquiry based on received inquiry information for inquiring whether a side effect caused by the drug taken has occurred in the person to be medicated at an occurrable timing determined based on the intake information at which the side effect is likely to occur, and transmit response information that is input in response to the inquiry and indicates whether the side effect has occurred.

A method is performed by a computer for adjusting an intake target of a drug indicating an amount of the drug to be taken by a person to be medicated The method includes acquiring intake information of a drug taken by a person to be medicated based on an intake target of the drug, determining, based on the intake information, an occurrable timing at which a side effect caused by the drug taken is likely to occur, presenting inquiry information for inquiring whether a side effect has occurred in the person to be medicated at the occurrable timing, acquiring response information that is input in response to the inquiry and indicates whether a side effect has occurred, changing the intake target of the drug based on the response information, and presenting the intake target changed.

The method may be performed by a computer for adjusting an intake target indicating an amount of a drug to be taken by a person to be medicated The method includes receiving intake information of a drug taken by a person to be medicated, determining, based on the intake information, an occurrable timing at which a side effect caused by the drug taken is likely to occur, transmitting information for presenting inquiry information for inquiring whether a side effect has occurred in the person to be medicated at the occurrable timing, receiving response information that is input in response to the inquiry and indicates whether a side effect has occurred, changing an intake target of the drug based on the response information, and transmitting information for presenting the intake target changed.

A method may also be performed by a computer for adjusting an intake target indicating an amount of a drug to be taken by a person to be medicated The method includes transmitting intake information of a drug taken by a person to be medicated input, presenting an inquiry based on received inquiry information for inquiring whether a side effect caused by the drug taken has occurred in the person to be medicated at an occurrable timing determined based on the intake information at which the side effect is likely to occur, and transmitting response information that is input in response to the inquiry and indicates whether the side effect has occurred.

A program causes a computer to perform the method described above.

It is thus possible to adjust intake of a drug in accordance with a person to be medicated based on occurrence information of a side effect caused by taking the drug.

### BRIEF DESCRIPTION OF THE DRAWINGS

FIG. 1 is a configuration diagram of a system according to an example.

FIG. 2 is a hardware configuration diagram of a user terminal, a server, and a medical practitioner terminal according to an example.

FIG. 3 is a functional block diagram of the user terminal and the server according to an example.

FIG. 4 is a flowchart according to an example.

FIG. 5 is a flowchart of an intake target determination process according to an example.

FIG. 6 is an example of a user terminal screen according to an example.

FIG. 7 is an example of a user terminal screen according to an example.

FIG. 8 is an example of a user terminal screen according to an example.

FIG. 9 is an example of a user terminal screen according to an example.

FIG. 10 is a flowchart of an intake target determination process according to an example.

FIG. 11 is a conceptual diagram of elapsed times with respect to plasma drug concentrations according to one example.

### REFERENCE SIGNS LIST

100 System
110 Network
120 User terminal
121 Processor
122 Display device
123 Input device
124 Storage device
125 Communication device
128 Bus
130 Server
131 Processor
132 Display device
133 Input device
134 Storage device
135 Communication device
138 Bus
140 Medical practitioner terminal
141 Processor
142 Display device
143 Input device
144 Storage device
145 Communication device
148 Bus
321 Input unit
322 Display unit
323 Communication unit
324 Control unit
325 Storage unit
331 Input unit
332 Display unit
333 Communication unit
334 Control unit
335 Side effect occurrable timing determination unit
336 Side effect inquiry information generation unit
337 Intake target determination unit
338 Storage unit
400 Intake target adjustment process

### DETAILED DESCRIPTION

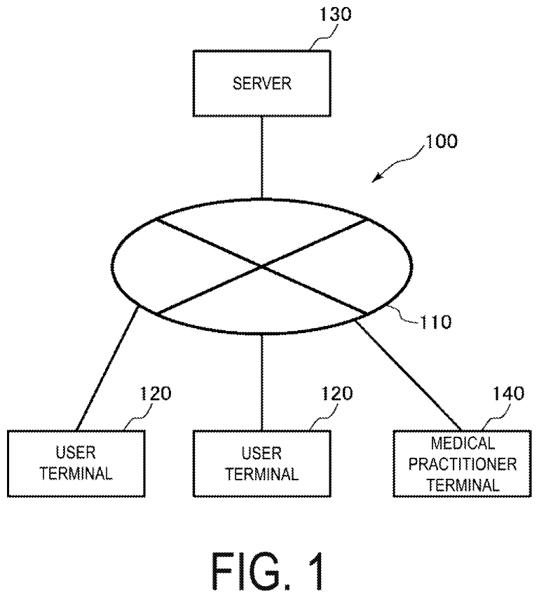

FIG. 1 illustrates an example of a system configuration diagram. A system 100 is used for gaining efficacy from intake of a drug, and includes a network 110, and a user terminal 120, a server 130, and a medical practitioner terminal 140 connected to the network.

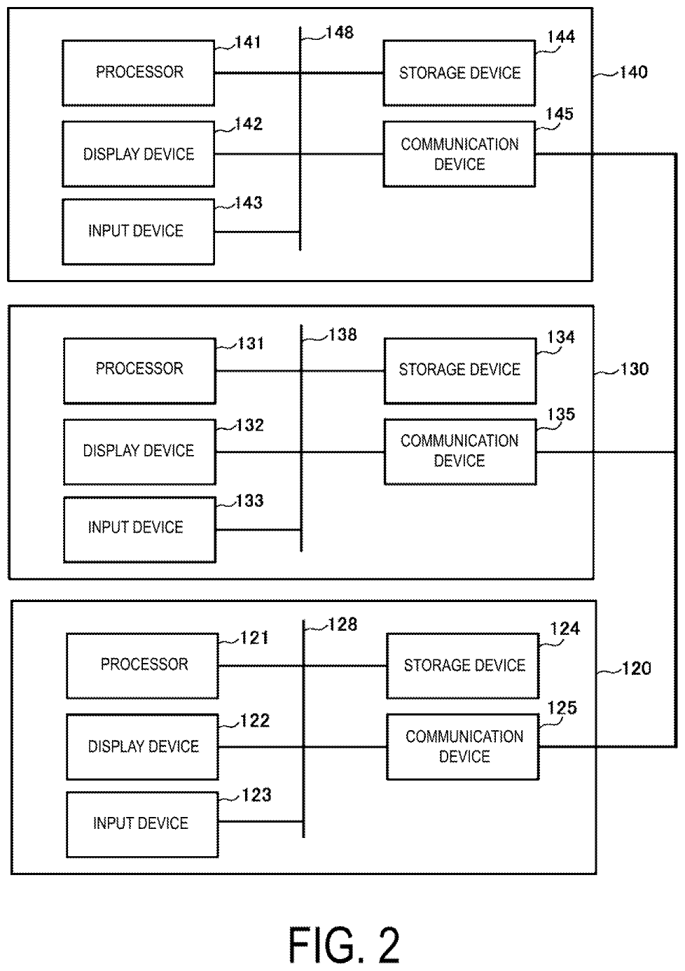

FIG. 2 is a block diagram illustrating a hardware configuration of the user terminal 120, the server 130, and the medical practitioner terminal 140 according to an example. The user terminal 120 includes a processor 121, a display device 122, an input device 123, a storage device 124, and a communication device 125. Each of these constituent devices is connected by a bus 128. Further, it is assumed that an interface is interposed between the bus 128 and each of the constituent devices as necessary. In this example, the user terminal 120 is a smartphone. However, the user terminal 120 can be an electronic device such as a tablet-type computer or a computer including a contact-type input device such as a touch pad as long as the user terminal has the configuration described above.

The server 130 also similarly includes a processor 131, a display device 132, an input device 133, a storage device 134, and a communication device 135. Each of these constituent devices is connected by a bus 138. Further, it is assumed that an interface is interposed between the bus 138 and each of the constituent devices as necessary. In this example, although the server 130 is implemented by a computer, other electronic devices may also be adopted.

The medical practitioner terminal 140 also similarly includes a processor, 141, a display device 142, an input device 143, a storage device 144, and a communication device 145. Each of these constituent devices is connected by a bus 148. Further, it is assumed that an interface is interposed between the bus 148 and each of the constituent devices as necessary. In this example, the server 130 is implemented by a computer. However, the medical practitioner terminal 140 can be an electronic device such as a tablet-type computer or a computer including a contact-type input device such as a touch pad as long as the medical practitioner terminal has the configuration described above.

The processors 121, 131, and 141 control all operations of the user terminal 120, the server 130, and the medical practitioner terminal 140, and are, for example, a CPU. The processors 121, 131, and 141 execute various types of processing by reading and executing a program and data stored in the storage devices 124, 134, and 144. In an example, the processors 121, 131, and 141 are constituted by a plurality of processors.

The display devices 122, 132, and 142 display an application screen or the like to the user of each of the device s under the control of the processors 121, 131, and 141. Although the display devices are preferably a liquid crystal display, they may be a display using organic EL, a plasma display or the like.

The input devices 123, 133, and 143 are user interfaces that receive inputs from the user with respect to the user terminal 120, the server 130, and the medical practitioner terminal 140, and are, for example, a touch panel, a touch pad, a keyboard, or a mouse. Since the user terminal 120 is a smartphone in this example, the user terminal 120 includes a touch panel as the input device 123, the touch panel also functions as the display device 122, and the display device 122 and the input device 123 have an integrated structure. The display device 122 and the input device 123 may be in separate forms in which the display device and the input device are disposed at separate positions. Because the server 130 and the medical practitioner terminal 140 are computers, the server and the medical practitioner terminal are assumed to each include a keyboard and a mouse as the input device, and a liquid crystal display as the display device.

The storage devices 124, 134, and 144 are storage devices provided in typical smartphones and computers, the storage devices including a storage device, a magnetic storage device, or the like using a RAM as a volatile memory and a flash memory such as an eMMC, a UFS, or an SSD as a nonvolatile memory. The storage devices 124, 134, and 144 can also include an external memory. For example, the storage devices 124 and 144 store a user application for performing this example, and the storage device 134 stores a server application.

The user application includes a user program for transmitting and/or receiving information to/from the server 130 and presenting information to a user, and various types of data referred to during execution of the user program. The user program is started in response to an operation of the user with respect to the user terminal 120 and the medical practitioner terminal 140, and is executed on an operating system (OS) implemented in advance by the user terminal 120 and the medical practitioner terminal 140. An application for the server includes a function and various types of data for enabling reception of an input from the user and presentation of information to the user to be suitably executed by the user program in each of the user terminal 120 and the medical practitioner terminal 140 that is a client.

The communication devices 125, 135, and 145 transmit and/or receive data to and/or from other devices via the network 110 (omitted in FIG. 2). For example, the communication devices 125, 135, and 145 perform mobile communication and wireless communication via a wireless LAN or the like, and are connected to the network 110. The user terminal 120 and the medical practitioner terminal 140 use the communication devices 125 and 145, respectively, to perform communication with the server 130 via the network. The communication devices 125, 135, and 145 may perform wired communication using an Ethernet (registered trademark) cable or the like.

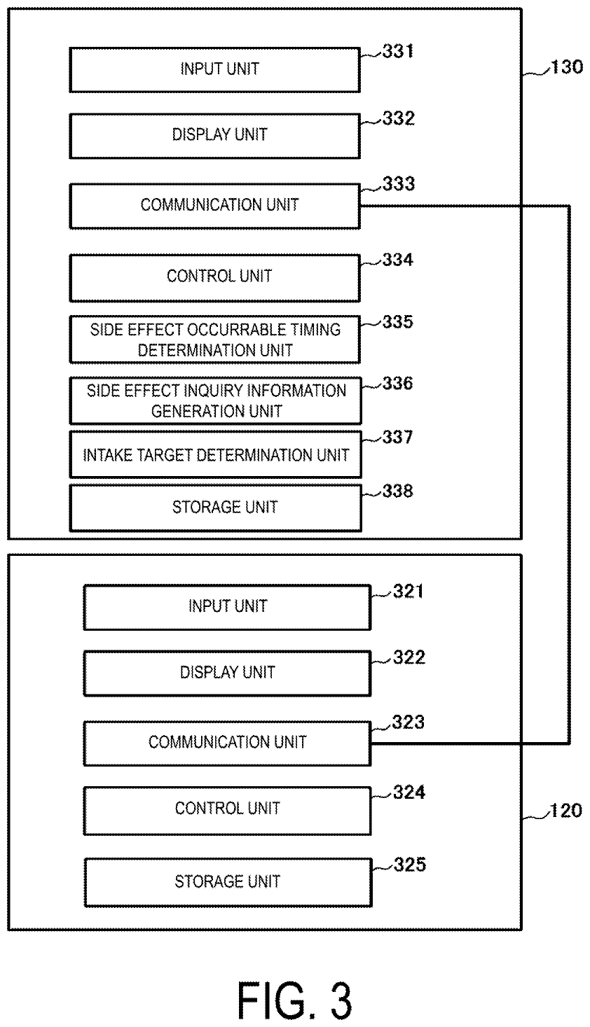

FIG. 3 illustrates an example of a functional block diagram of the user terminal 120 and the server 130. In this example, it is assumed that functional blocks of the medical practitioner terminal 140 are the same as those of the user terminal 120, and illustration and description thereof are omitted. The user terminal 120 includes an input unit 321, a display unit 322, a communication unit 323, a control unit 324, and a storage unit 325, and the server 130 includes an input unit 331, a display unit 332, a communication unit 333, a control unit 334, a side effect occurrable timing determination unit 335, a side effect inquiry information generation unit 336, an intake target determination unit 337, and a storage unit 338. The input units 321 and 331 each have a function of receiving inputs from a user.

The display units 322 and 332 each have a function of displaying information to be visible to a user. The communication units 323 and 333 each have a function of transmitting and/or receiving information to and/or from other devices. The control units 324 and 334 each have a function of executing control of information processing, or the like. The control units 324 and 334 store and update data in the storage units 325 and 338. The storage units 325 and 338 each have a function of storing tables, data and the like.

The side effect occurrable timing determination unit 335 has a function of determining a timing at which a side effect is likely to occur in the person to be medicated who is a user. The side effect inquiry information generation unit 336 has a function of generating information for inquiring to the person to be medicated whether a side effect that is likely to occur in the person has been expressed based on information stored in the storage unit 338. The intake target determination unit 337 has a function of determining an intake target of the drug based on medical history data stored in the storage unit 338 and information of the response to the inquiry from the user about whether a side effect has occurred. In this example, these functions are achieved by causing programs stored in the storage devices 124 and 134 to be executed by the processors 121 and 131 described in FIG. 2 to operate respective hardware and software in cooperation, and the functions can also be implemented by configuring an electronic circuit or the like for achieving each of the functions.

Although, when a patient who takes a therapeutic drug for smoking cessation treatment under instructions of a medical practitioner is set as a person to be medicated (user) will be described in this example, a person to be medicated does not need to be instructed by a medical practitioner, and a person to be medicated (user) who takes any kind of drug may be applicable. A drug is not limited to a prescription drug intended to treat medical diseases such as disorder of gastric function, hypertension, and mental illness, and includes a drug that is expected to bring favorable effects on the health of the person to be medicated such as a drug taken to improve, enhance, or maintain health habits or health conditions. The drug includes supplements. In addition, a method of taking a drug is not limited to oral administration, and any method may be used as long as it is a method of taking a drug into the body. A side effect is not limited to a side effect caused by a drug, and includes an effect occurring when the drug is taken, which is inappropriate for the purpose of the drug or an effect adversely acting on a living body.

Although information about the history of the user is stored in the storage unit 338 of the server in this example, a part or all of the information may be stored in the storage unit 325 of the user terminal 120 and the storage unit of the medical practitioner terminal 140. The information about the history of the user includes attribute data, prescription drug data, dosing data, drug efficacy data, and wake-up time data of the user (patient). Examples of user attribute data and prescription drug data are shown in Tables 1 and 2.

TABLE 1
User Attribute Data

| User ID | Name | Date of birth | Age | Start date |
|---|---|---|---|---|
| 1 | Patient A | Jul. 3, 1978 | 40 | Apr. 23, 2020 |
| 2 | Patient B | Jan. 3, 1987 | 30 | Apr. 28, 2020 |
| . . . | . . . | . . . | . . . | . . . |

The user attribute data includes name, date of birth, age, and treatment start date of users. These pieces of information are stored in association with user IDs. Age may be updated at a predetermined timing based on the date of birth. Start date is the date on which treatment starts, and may be, for example, the date on which a user receives an outpatient care for the first time.

TABLE 2
Prescription Drug Data

| | | | | Total | | Number |
|---|---|---|---|---|---|---|
| Data | Drug | | User | amount | Dose | of doses |
| ID | ID | Drug name | ID | (mg) | (mg) | per day |
| 1 | 1 | Smoking | 1 | 70 | 1.0 | 2 |
| | | cessation aid A | | | | |
| 2 | 1 | Smoking | 1 | — | 1.5 | 2 |
| | | cessation aid A | | | | |
| . . . | . . . | . . . | . . . | . . . | . . . | . . . |

The prescription drug data includes data ID, drug ID, prescription drug name, user ID, a total amount, dose, and the number of doses per day. A drug ID is an ID assigned to each drug, and although omitted here, a drug table may be separately prepared in advance to store drug IDs and drug names in association with each other. For example, when a doctor refers to a drug table and selects and inputs a drug to be prescribed for a patient, the drug is stored in the storage unit 338 of the server as prescription drug data. The total amount indicates the total amount of drug prescribed to the patient and is determined by a medical practitioner. In this example, there is a possibility of a dosage of a drug being insufficient if a total amount of the drug is determined only based on an initial value of an intake target indicating the amount of drug to be taken by a patient because the intake target varies. Therefore, an appropriate total amount is determined by a medical practitioner so that dosing can be continued even when the intake target is adjusted. A dose indicates an amount of target intake per time, and the number of doses per day indicates the number of intakes per day. Two times mentioned here means that a patient has to take the drug twice after breakfast and dinner. In addition, information indicating whether the drug should be taken before or after a meal may be included. Tables 1 and 2 can be input by using the medical practitioner terminal 140 used by the doctor and stored in the storage unit 338 of the server when the user receives an outpatient care of the doctor who is a medical practitioner and starts smoking cessation treatment.

As one modified example, with a drug which is sold without an instruction of a medical practitioner, a person to be medicated may be able to purchase the drug in a pharmacy or the like, install a predetermined application in the user terminal 120, performs user registration to the server 130, and register user attribute data and prescription drug data as shown in Tables 1 and 2. The dose and the number of doses per day may be set as specified amounts as initial values determined by the manufacturer of the drug, and may be automatically input at the time of user registration.

The storage unit 338 of the server further stores a side effect table as shown in Table 3. The side effect table shows side effects that is likely to occur when each drug is taken and an occurrence probability thereof. Furthermore, a probability of occurrence of a side effect may vary under certain conditions. For example, although drowsiness as a side effect of the smoking cessation aid A (drug ID=1) has a normal occurrence probability of 5% or higher, during the period of 8 hours from the wake-up time to 10 hours from the wake-up time, it has an occurrence probability 5% higher than the normal occurrence probability, that is, 10% or higher. The variation probability “+5%” in the data in the first row of Table 3 indicates that the occurrence probability is +5% when the variation condition is satisfied, and “t=8 hours to 10 hours from the wake-up time” in the variation condition means that the variation condition is satisfied when a time (timing) t at which side effect inquiry information is presented is in 8 hours to 10 hours from the wake-up time. The variation probability and the variation condition for a side effect whose occurrence probability does not vary are expressed with “−.”

TABLE 3
Side Effect Table

| Drug | | | Variation | Variation |
|---|---|---|---|---|
| ID | Side effect | Probability | probability | condition |
| 1 | Drowsiness | 5% or higher | +5% | t = 8 hours to |
| | | | | 10 hours from |
| | | | | wake-up time |
| 1 | Nausea | 0.5% or higher | — | — |
| | | Less than 5% | | |
| 1 | Insomnia | 5% or higher | — | — |
| 1 | Abnormal | 5% or higher | — | — |
| | dreams | | | |
| 1 | Headache | 5% or higher | — | — |
| 1 | Abdominal | 5% or higher | — | — |
| | distention | | | |
| 1 | Constipation | 5% or higher | — | — |
| 1 | Gastritis | Less than 0.5% | — | — |
| 1 | Vomiting | 5% or higher | — | — |
| 2 | Nausea | 3% or higher | — | — |
| . . . | . . . | . . . | — | — |

The information indicating the medical treatment history of the user includes daily wake-up time, presence or absence of drug efficacy (drug effect), and dosing information. The dosing information is intake information of a drug taken by a patient and includes the ID of the taken drug, dosing time, and a dosage. Table 4 shows medical treatment history data as an example used in this example.

TABLE 4
Medical Treatment History Data

| Data | Wake-up | Drug | Dosing information | | |
|---|---|---|---|---|---|
| ID | time | efficacy | Drug ID | Dosing time | Dosage |
| 1 | 06:30 | 0 | 1 | 08:00 Apr. 25, 2020 | 1.0 mg |
| 2 | — | 0 | 1 | 19:00 Apr. 25, 2020 | 1.0 mg |
| 3 | 07:00 | 0 | 1 | 08:10 Apr. 26, 2020 | 1.0 mg |
| . . . | . . . | . . . | . . . | . . . | . . . |
| 15 | 06:00 | 0 | 1 | 08:00 May 1, 2020 | 1.0 mg |
| 16 | — | 0 | 1 | 19:20 May 1, 2020 | 1.5 mg |
| 17 | 06:00 | 0 | 1 | 08:00 May 2, 2020 | 1.5 mg |
| . . . | . . . | . . . | . . . | . . . | . . . |

The data shown in Table 4 is medical treatment history data associated with the user ID of one user. The data ID is information for identifying each of a plurality of pieces of data associated with the user ID. In this example, the user can input medical treatment history data via a diary function of an application executed in the user terminal 120 for carrying out this disclosure. The wake-up time is a wake-up time of the user on that day. Since the wake-up time may be input once a day, the input is omitted at data ID=2. The drug efficacy is information indicating whether the user feels the medical effect at the time of input. “Efficacy=0” indicates that the efficacy is not felt, and “efficacy=1” indicates that the efficacy is felt. The dosing information includes information about daily dosing, the drug ID indicates the ID of the drug taken, the dosing time indicates the time at which the drug is taken, and the dosage indicates the amount of the drug taken based on the intake target.

Although these pieces of information are manually input by the user in this example, the information may be automatically input using a sensor. The input information may be biological information such as blood pressure. For example, the wake-up time may be automatically determined by a wearable terminal including a sensor, or the blood pressure of a hypertension patient may be automatically measured to determine whether the drug is effective and input the information.

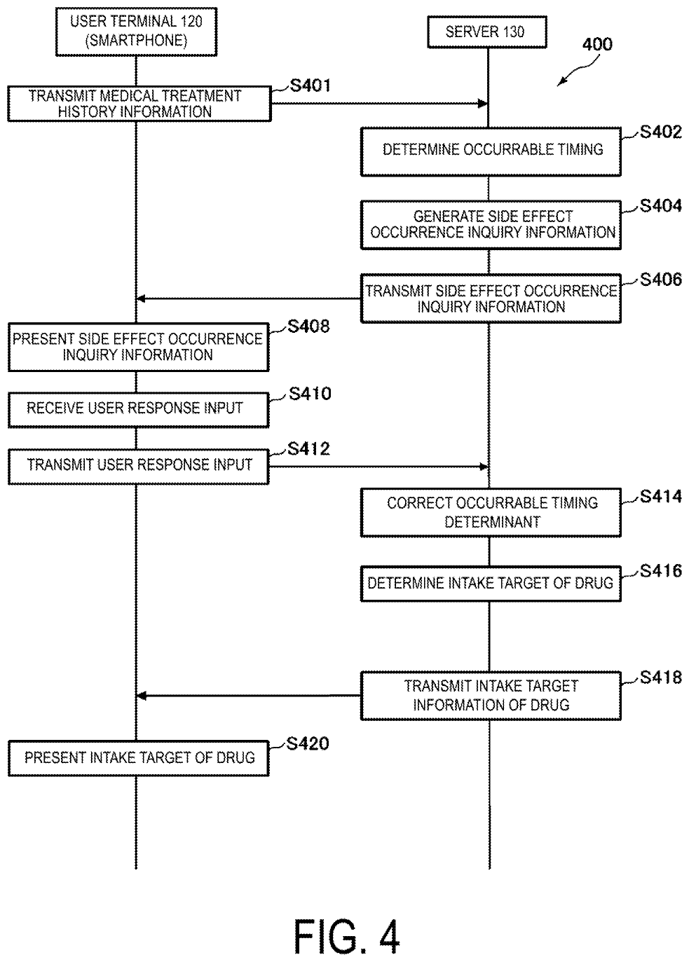

Next, an operation of the system in this example will be described with reference to FIG. 4. Although an operation of one user terminal 120 will be described for explanation in this example, the example may include two or more user terminal 120.

When a patient is a patient A who wants to stop smoking and visits as an outpatient to a medical practitioner, if the medical practitioner transmits information for registration as a user via the medical practitioner terminal 140, the information is stored as user attribute data in the server 130 as shown in Table 1. Furthermore, a smoking cessation aid A is prescribed to be taken twice a day (after breakfast and dinner), with 1.0 mg/dose as an initial intake target. Since the dosage may be increased after the start of dosing, the drug is prescribed with the maximum dose of 2.5 mg twice a day, and the maximum amount for two weeks of 70 mg. This maximum dose is set in advance by, for example, the medical practitioner or the drug manufacturer. When these pieces of information are input to the medical practitioner terminal 140 by the medical practitioner, they are transmitted to the server 130 and stored as prescription drug data (Table 2).

The patient A who is a user downloads and installs an application necessary for implementing this example in his or her user terminal 120. At this time, the user inputs his or her user ID in the application to store the user ID in the storage unit 325 of the user terminal. After that, the user uses the diary function of the installed application to input medical treatment history information after taking the drug every day. The medical treatment history information includes wake-up time, presence or absence of efficacy, a drug taken according to the intake target, time, and an amount. When the medical treatment history information is input, information indicating the input data is transmitted to server 130 together with the user ID, and is stored in the server 130 as medical treatment history data (Table 4) of the user.

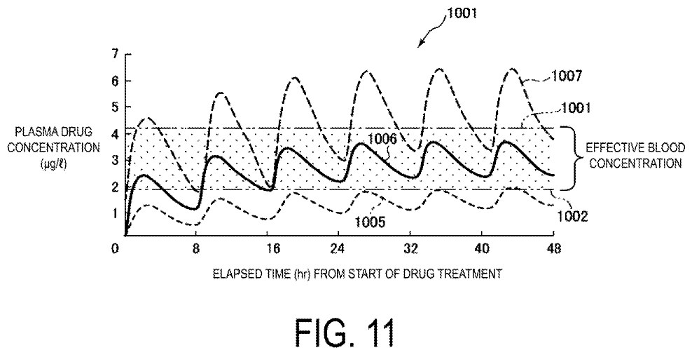

In this example, the dosage determined by the medical practitioner at the time of the outpatient care is maintained for a certain period from the start of dosing, and then an adjustment process for the intake target is started. As shown in FIG. 11, the plasma drug concentration after taking the drug is stabilized after a certain period of time, allowing the effects and side effects of the drug to be more stably observed after dosing is continued for a certain period of time. It is assumed that the patient A is medicated with 1.0 mg of the smoking cessation aid A after breakfast and dinner for one week and the dosing information and efficacy information are recorded in the server 130 by using the diary function of the application.

The efficacy information can be acquired by causing the user to input whether there is a withdrawal symptom. For example, when the application receives a user input for activating the diary function, the application presents information for inquiring whether a withdrawal symptom has appeared to the user. The user can input information indicating whether efficacy has occurred by responding with “yes” or “no” thereto. For “yes,” the withdrawal symptom appears, since the efficacy has not sufficiently occurred, and “0” is set for “efficacy” in the medical treatment history data. For “no,” no withdrawal symptom has appeared, and thus the efficacy has sufficiently occurred, and “1” is set for “efficacy.” In addition, the patient is assumed to input his or her wake-up time in input to the diary after breakfast.

Then, after one week elapses from the start of dosing, the intake target adjustment process 400 shown in FIG. 4 is repeatedly executed once a day. Although it is assumed that the intake target adjustment process is started every day with first transmission of medical treatment history information as a trigger, the intake target adjustment process may be started in another method such as starting the process at a predetermined time. When the intake target adjustment process is started at a predetermined time, for example, the medical treatment history information input last can be read from the storage unit 338 of the server 130 and used as medical treatment history information.

When the patient A takes a drug every morning, the patient A uses the diary function of the application to input medical treatment history information including information indicating the wake-up time, the presence or absence of efficacy, the taken drug, the time, and the amount through the user terminal 120 (S401). Upon receiving the dosing information, the server 130 determines the occurrence timing of side effect based on the medical treatment history information (S402). The occurrence timing of side effect is determined based on dosing time, an occurrable timing determinant, and a correction value. The occurrable timing determinant is the estimated time to a maximum plasma concentration (Tmax). The occurrable timing of side effect may be a side effect occurrable period with a certain length or may be specific time having no period. The correction value is a value for correcting the occurrable timing determinant in accordance with a patient. Although the initial value is set to zero here, the initial value may be given in advance based on user attribute information or the like.

Next, the server 130 generates side effect occurrence inquiry information (S404). In this example, the side effect occurrence information includes information indicating a side effect that can occur in the patient and a probability of occurrence of each side effect and information indicating a variation probability, a variation condition, and an occurrable timing. The side effect table (Table 2) stored in the storage unit 338 of the server 130 can be used for the probability of occurrence of a side effect.

When there is a plurality of side effects that are likely to occur, priority order information for display is included, and a side effect a higher priority order can be preferentially displayed on the user terminal 120. When a display unit is small like a smartphone, a plurality of side effects may not be displayed at the same time in some instances. Images of side effects are displayed in descending order of priority, and those which cannot be displayed can be displayed by scrolling or the like. A priority order can be determined based on, for example, the probability of occurrence of a side effect or the severity of the side effect on health. The user terminal 120 may determine a priority order based on an occurrence probability.

The server 130 transmits the generated side effect occurrence inquiry information to the user terminal 120 (S406). The user terminal 120 presents a side effect occurrence inquiry to the user based on the occurrable timing included in the side effect occurrence inquiry information (S408). When the side effect occurrable timing is a side effect occurrable period having a certain length, the user terminal 120 presents information for inquiring whether a side effect has occurred during the period. The information does not need to be continuously presented at all times during the side effect occurrable period.

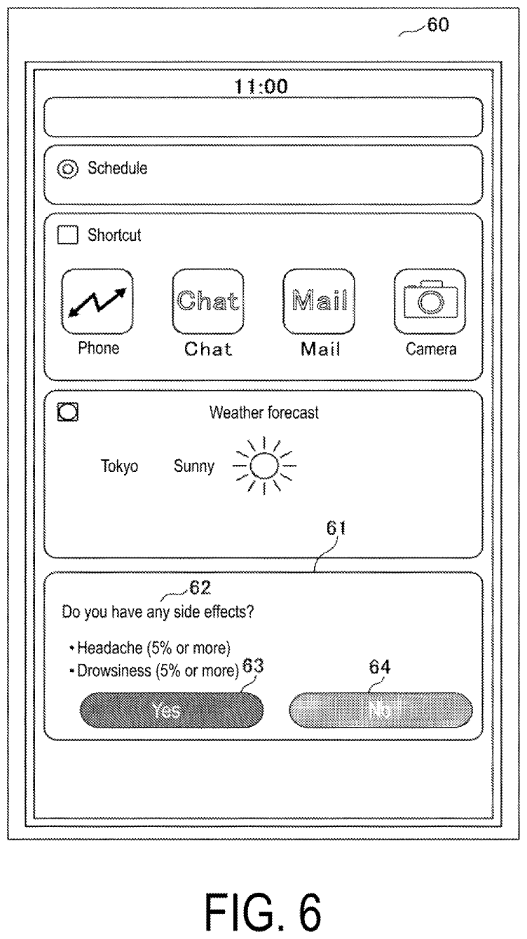

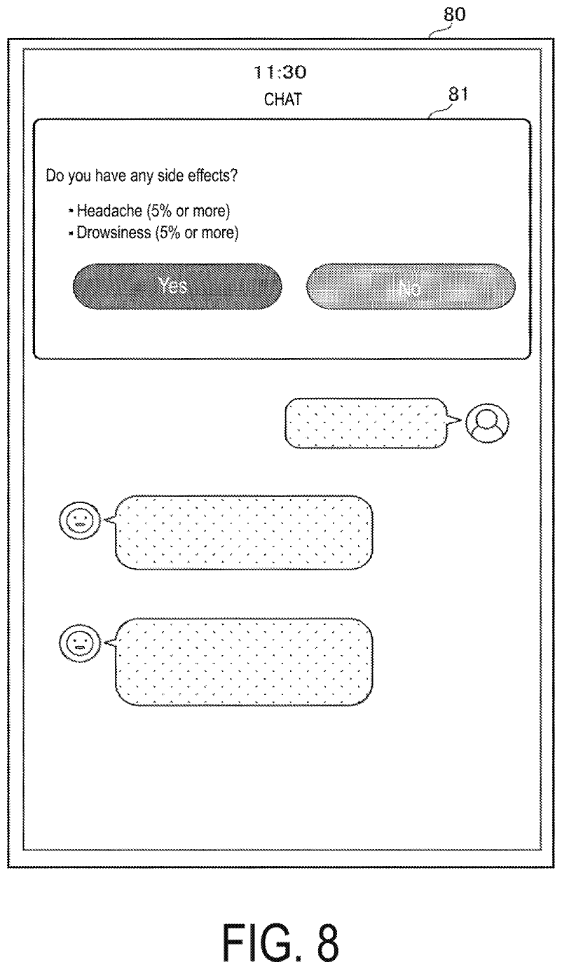

For example, the side effect inquiry information can be displayed in the notification area of the application as illustrated in FIG. 6. Even if the user does not activate the application of this disclosure, if the time in which the user operates the smartphone and views the notification display areas of the plurality of applications is a side effect occurrable period, a side effect occurrence inquiry can be made. Furthermore, the side effect inquiry information may be displayed on an application screen displayed when the application is activated as illustrated in FIG. 8. A timing at which the side effect occurs varies among individuals and is not necessarily uniquely fixed even for the same person due to physical conditions or the like. Occurrence of a side effect can be detected without omission by enabling presentation of information of inquiring the occurrence of a side effect during a period having a certain span. Even during a period having a certain span, it is not necessary to display the side effect inquiry information while another application is being used or when the notification display area of the application is not displayed.

During the side effect occurrence inquiry period, an inquiry may be made a plurality of times even after the response indicating response information that no side effect occurs is input. Although no side effect occurs in the initial stage of the side effect occurrence inquiry period, a side effect may occur later. Making a plurality of inquiries within the inquiry period can easily detect a side effect that occurs later. In an inquiry made a plurality of times, S414 to S420 may be performed each time a response input is received, or S414 to S420 may be performed when a response input indicating that a side effect has occurred is received.

Furthermore, a side effect occurrable timing may be a specific time which does not have a certain length. In this example, at a specific time that is the side effect occurrable timing, the user terminal 120 can be made to sound a ringtone, activate a vibration function, or the like, and present a side effect occurrence inquiry. Occurrence of a side effect can be detected by positively making an inquiry to the patient at a timing at which the side effect is highly likely to occur. The specific time does not need to be provided one time. A plurality of specific times may be set and inquiry information may be presented at each of the times, S414 to S420 may be performed each time a response input is received, or S414 to S420 may be performed when a response input indicating that a side effect has occurred is received.

The side effect occurrable timing can be either a period with a certain length or a specific time without a certain length or may be both. Allowing a side effect occurrence inquiry to be presented during the period with a certain length and actively inquiring about a side effect occurrence by sounding a ringtone at a specific time allows the patient to operate the user terminal 120 during the side effect occurrable period and respond to the inquiry at the timing when viewing the side effect occurrence inquiry. Even when the patient does not operate the user terminal 120 during that period, the side effect occurrence can be detected more reliably by actively making an inquiry at the specific time.

When the presented side effect has occurred, the user enters an input indicating that the side effect has occurred by touching “occurred” (63), and when no side effect occurs, the user enters an input indicating that no side effect occurs by touching “not occurred” (64). When no response input is made for a predetermined period after the inquiry information is presented to the user, the intake target adjustment process 400 may be ended, or the subsequent process may be executed by regarding that there is a response input indicating that no side effect occurs.

Upon receiving the response input from the user (S410), the user terminal 120 transmits the response input to the server 130 (S412). The server 130 performs a side effect occurrable timing determinant correction process based on the received user response input (S414). For example, based on the assumption that an occurrable timing determinant is an estimated time to a maximum plasma concentration (Tmax) and that the time at which a response input from the user indicating occurrence of the side effect is an actual Tmax, a correction value of the occurrable timing determinant can be determined. The determined occurrable timing determinant correction value is used in the next and subsequent occurrable timing determination process (S402).

Thereafter, an intake target determination process for a drug is executed based on a response input from the user (S416). In the intake target determination process, for example, the intake target is adjusted such that the plasma drug concentration of the patient converges to the effective blood concentration between the line 1001 and the line 1002 in FIG. 11. FIG. 11 shows the time transition of the plasma drug concentration of a patient who took the drug every 8 hours. Line 1005 indicates when the plasma drug concentration is equal to or lower than the effective blood concentration, line 1007 indicates when the plasma drug concentration is equal to or higher than the effective blood concentration, and line 1006 indicates when the plasma drug concentration is within the effective blood concentration.

When the plasma drug concentration exceeds the effective blood concentration, a side effect may occur, adversely affecting the body of the patient. On the other hand, when the plasma drug concentration is lower than the effective blood concentration, the efficacy is not sufficiently exhibited. The plasma drug concentration increases after the drug is taken to the time to a maximum plasma concentration (Tmax), then decreases, and increases with the next intake of the drug in a repeating manner. In this example, the intake target is adjusted to the intake of the drug in which the plasma drug concentration converges to the effective blood concentration based on the side effect occurrence information and the efficacy occurrence information of the patient. The intake target determination processing (S416) may use any method such as a linear search method or a binary search method as long as it is a method of reaching an appropriate intake target.

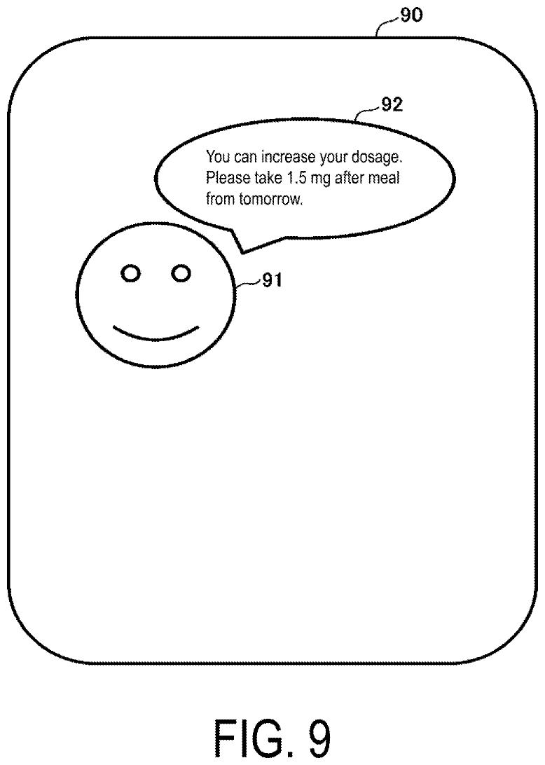

When the intake target of the drug is changed, information indicating the changed intake target is transmitted to the user terminal 120 (S418), and the user terminal 120 that has received the information presents the new intake target to the user (S420) as shown in FIG. 9, for example, and ends the intake target adjustment process. In this example, the process from S401 to S420 is performed once a day and is repeatedly performed during the treatment period.

As a modified example, when the intake target of the drug is changed, the changed intake target information may be transmitted to the medical practitioner terminal 140 (not illustrated) in addition to or instead of the user terminal 120. The medical practitioner can monitor the dosage of the patient. When the doctor determines that the changed intake target is not appropriate, the doctor may be able to input a re-change instruction via the medical practitioner terminal 140. For example, when a new intake target deemed appropriate is input by the medical practitioner to the medical practitioner terminal 140 and transmitted to the server 130, the server 130 updates the prescription data (Table 2) based on the new intake target and notifies the user terminal 120 of the changed intake target.

In addition, the medical treatment history information and the user response input with respect to the side effect occurrence inquiry transmitted from the user terminal 120 in the S401 and the S412 may also be transmitted to the medical practitioner terminal 140. An intake target which is different from the intake target of the drug changed by the server 130 but is considered to be more appropriate by the medical practitioner can be set based on these pieces of information. Furthermore, a target change process more suitable for the patient can be performed by resetting a constant or the like used for determination by the medical practitioner to change the intake target of the drug.

An intake target to be changed is not limited to intake per dosage. The number of intakes per day may be changed. For example, the number of intakes which is twice a day after breakfast and dinner may be changed to three times a day after each meal.

An example of process using this example will be described in more detail by using specific numerical values. Content overlapping with the above description will be omitted. The patient A to be treated for smoking cessation received medical treatment by having an interview with the doctor and was prescribed with the smoking cessation aid A. The initial intake target was set to 1.0 mg of the drug to be taken twice a day after breakfast and dinner. On May 1, the eighth day of the dosing, the patient took 1.0 mg of the drug at 08:00 after breakfast according to the intake target. However, the patient A has not been cured of the withdrawal symptom (abstinent symptom). Thereafter, at 08:30, the patient input dosing information indicating that 1.0 mg was taken, efficacy information indicating that there was a withdrawal symptom, and wake-up time information indicating that the wake-up time was 06:00 using the diary function of the application. The user terminal 120 transmits the input information as medical treatment history information to the server 130 (S401). The server 130 receives the medical treatment history information and records it in association with the user ID of the user A as medical treatment history information.

The server 130 determines the side effect occurrable timing based on the dosing information included in the received medical treatment history information. In this example, the estimated time to a maximum plasma concentration (Tmax) is used as an occurrable timing determinant for determining the side effect occurrable timing. Then, the period from 30 minutes before the estimated Tmax to 4 hours after the estimated Tmax is assumed to be a side effect occurrable timing. Although it is assumed that the estimated Tmax is stored in advance in the server 130 for each dosage of the smoking cessation aid A, the estimated Tmax may be determined by calculation based on the drug being taken, the dosage and the like.

The side effect occurrable timing determination unit 335 of the server 130 reads from the storage unit 338 that the estimated Tmax when 1.0 mg of the smoking cessation aid A is taken is 3 hours and determines the period from 30 minutes before to 4 hours after the estimated Tmax as a side effect occurrable timing. As the estimated Tmax as the initial value, a standard estimated Tmax determined in advance as a general value is used. The dosing time is 08:00, and a period from 10:30 to 15:00 is set as the side effect occurrable timing. The side effect occurrable timing may be set to 11:00 that is the estimated Tmax.

The server 130 reads the side effect of the smoking cessation aid A and the occurrence probability of each side effect from the side effect table (Table 3) stored in the storage unit 338, generates side effect occurrence inquiry information indicating them, and transmits the side effect occurrence inquiry information to the user terminal 120.

The user terminal 120 that has received the side effect occurrence inquiry information presents the side effect occurrence inquiry information on the display unit 322 based on the side effect occurrence inquiry information. The inquiry information is presented in response to the user turning on the screen display of the user terminal 120 and performing a user input indicating a presentation request for requesting presentation of a notification display from the application from 10:30 to 15:00 which is the period determined to be the side effect occurrable timing. For example, as notification information (61) from the application for this example together with a notification from the other application, an inquiry “do you have a side effect?” (62) and headache (5% or more) and drowsiness (5% or more) having a high occurrence probability among the side effects that are likely to occur are displayed as illustrated in FIG. 6. Other side effects such as insomnia, abnormal dreams, and the like can also be displayed when the user scrolls the input, or the like. The user terminal 120 may sound a ringtone or activate the vibration function when the side effect occurrable timing starts to actively notify the user of the fact that there is a side effect inquiry.

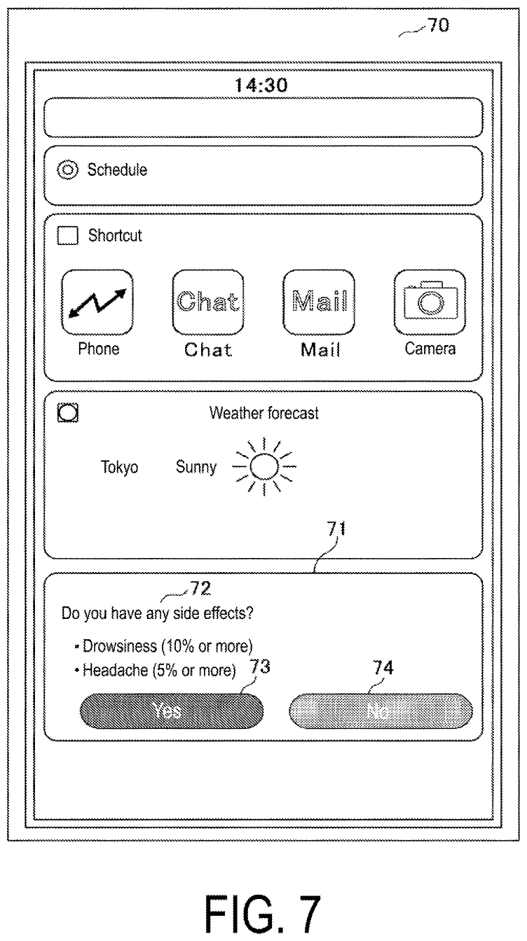

Furthermore, regarding drowsiness as a side effect of the smoking cessation aid A, “variation probability+5%” and “variation condition t=wake-up time+8 hours to 10 hours” are set in the side effect table (Table 3), which indicates that the side effect occurrence probability also varies depending on time slots. Therefore, it is possible to include the information indicating the variation probability and the variation condition in the side effect inquiry information, and cause the user terminal 120 to determine the varying occurrence probability based on that information and the wake-up time information included in the medical treatment history information and re-present the occurrence probability in descending order. Since the patient A wakes up at 06:00, the occurrence probability of drowsiness is +5% between 14:00 and 16:00, and the occurrence probability is 10% or higher between 14:00 and 15:00. As illustrated in FIG. 7, for example, in the inquiry information presented at 14:30, the drowsiness level is changed to 10% or higher and is displayed at the top because of its highest occurrence probability.

When the patient A inputs a response indicating that the side effect occurs in response to the inquiry, the server 130 determines the correction value of the estimated Tmax as the occurrable timing determinant based on the time at which the user response input is input (S414). Although the standard estimated Tmax is set to 3 hours, it is assumed that a user response input indicating that the side effect has occurred at 10:45 which is 2.75 hours later is received. In this example, it is assumed that −0.25 hours is set as a correction value of the estimated Tmax, and a value obtained by subtracting the correction value from the standard estimated Tmax is used as the estimated Tmax to be used from the next time.

Although a correction value is determined based on only the reception time of one response input, the average value of differences between a plurality of response inputs and the estimated Tmax may be used, or a correction value may be determined using another method. A occurrable timing of a side effect varies depending on individuals, and a side effect may not be detected when an occurrence timing is significantly different from an occurrable timing determined based on the standard estimated Tmax. Correcting the occurrable timing determinant allows the expression timing of the side effect to be adjusted to a timing suitable for each individual and a possibility of missed detection of a side effect occurrence to be reduced.

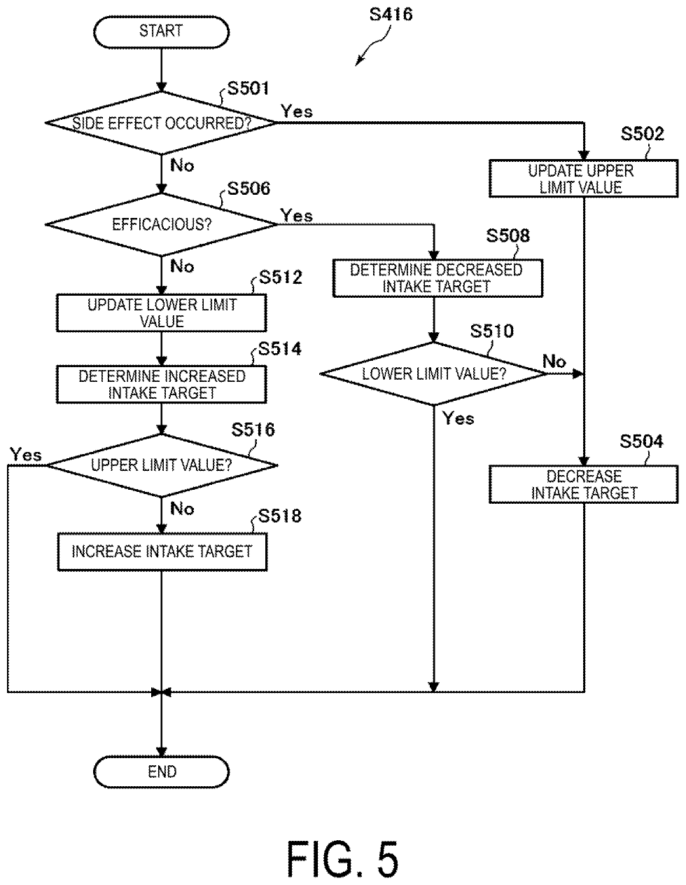

Next, an example of a process (S416) of changing an intake target of the drug will be described with reference to FIG. 5. It is determined whether a side effect has occurred based on the user response input indicating the presence or absence of the efficacy and the side effect of the drug transmitted from the user in the S412 (S501). If the side effect has occurred, the upper limit value of the intake target of the drug is updated by being changed to the current intake target (S502), and the dosage is reduced by the amount determined using a predetermined method (S504). If the intake target has been changed, the changed intake target information is registered in the prescription drug data stored in the storage unit 338 of the server 130. For example, when 1.0 mg is stored as “dose” in data ID=1 in Table 2 and the intake target is changed to 1.5 mg, the intake target of the user can be updated by storing 1.5 mg as “dose” in data ID=2. “Dose” of data ID=1 may be changed.

When no side effect occurs, it is determined whether efficacy has occurred based on the user response input (S506), and when efficacy has occurred, a reduced intake target of the drug is calculated (S508). It is determined whether the reduced intake target is equal to or lower than the lower limit value (S510). If the reduced intake target is determined not to be equal to or lower than the lower limit value, the dosage is reduced (S504), and if the reduced intake target is determined to be equal to or lower than the lower limit value, the intake target is not changed. If no efficacy occurs, the lower limit value of the intake target is updated by being changed to the current intake target (S512), and an increased intake target is determined. It is determined whether the increased intake target is equal to or higher than the upper limit value (S514), and if the increased intake target is not equal to or higher than the upper limit value, the intake target is increased (S516), and if the increased intake target is equal to or higher than the upper limit value, the intake target is not increased.

The initial values of the upper limit value and the lower limit value of the intake target can be set to values determined in advance by, for example, a medical practitioner or the like. Setting and updating the dosage (intake target) at which the side effect has occurred as the upper limit value in the intake target determination process can avoid adjustment to a dosage at which a side effect may occur. In addition, by setting the dosage at which no efficacy is felt as the lower limit value, occurrence of a symptom of a sickness such as withdrawal symptoms can be avoided, and thus a burden on the person to be medicated can be reduced.

Setting an intake target at which a side effect occurs as an upper limit value, setting an intake target at which no efficacy occurs as a lower limit value, and making no change exceeding the upper limit value or the lower limit value allows an intake target to be caused to converge on an appropriate intake target with a smaller number of changes. It is also possible to prevent a non-convergence state of an intake target by repeatedly changing the intake target between the intake target at which side effect occurs and the intake target at which no efficacy occurs. In this example, whereas when a side effect occurs (S501), the intake target is necessarily decreased (S504), when no efficacy occurs and the intake target is below the lower limit value, the intake target is not changed and processed to converge. Either the upper limit value or the lower limit value of the dosage does not need to be set, or neither of them may be set.

An example of a method of calculating a variation when the intake target is increased (increase) or decreased (decrease) will be described. It is assumed that the intake target of a drug after n times of change is A(n), and an n-th variation of the drug is v(n). An initial value A(0) of the intake target is determined by a medical practitioner. Although an initial variation amount v(1) is set to A(0)/2, it may be set to an amount predetermined by a medical practitioner or the like. A(n) in n>0 and v(n) in n>1 can be determined by formulas (1) and (2):

A(n)=A(n−1)+/−v(n−1) (1)

v(n)=rounddown(v(n−1)/2) (2)

“Rounddown” is an operator that means rounding down to the second decimal place. When v(n)=0.0 is obtained by rounding down to the second decimal place in Formula (1), v(n)=0.1 is set. In addition, the notation “+1-” in Formula (1) represents “−” when the user response input indicates that a side effect has occurred, and represents “+” when the user response input indicates that no side effect occurs.

In this example, it is determined that no side effect occurs based on the user response input of the patient A on May 1 (S501), and it is determined that no efficacy has been exhibited based on the medical treatment history information (S506). Since 1.0 mg of the dosage of that time that does not exhibit efficacy is indicated, 1.0 mg is set as the lower limit value of the intake target (S512). Since the change of the intake target is for the first time, the variation v(1) is calculated to be +0.5 mg that is a half of 1.0 mg of the initial value A(0)=1 of the intake target, and the intake target A(1) in the increase is calculated to be 1.5 mg (S514). It is determined that the intake target is less than 2.5 mg that is the initial upper limit value (S516), and the intake target calculated in S514 is increased (+0.5 mg) (S518).

The server 130 transmits the changed intake target information of the drug to the user terminal 120 (S418), and the user terminal 120 presents information for notifying the change of the intake target to the user (S420), and the intake target adjustment process of that day is ended. After dinner, the patient A takes 1.5 mg of the smoking cessation aid A, which is a new intake target, and inputs the medical treatment history information via the user terminal 120, and although the server 130 stores the medical treatment history information in the medical treatment history information, but does not execute the process for adjusting the intake target of the drug.

The patient A uses the diary function of the application to input to the user terminal 120 that he or she took 1.5 mg of the smoking cessation aid A at 08:00 in the morning, woke up at 06:00, and consecutively suffered from withdrawal symptoms on the next day, May 2. The user terminal 120 and the server 130 execute the processes of S401 to S420 based on these pieces of information. The same process as that of the previous day is omitted to avoid overlapping description.

Since a correction value of the occurrable timing determinant is determined to be −0.25 in the process of the previous day, the corrected estimated Tmax is 2.75 hours. Thus, the side effect occurrable timing is determined to be a time from 10:15 to 14:45 (S402). Similar to the previous day, it is assumed that the patient A inputs to the user terminal 120 that no side effect occurs. The server 130 determines that no side effect occurs (S501), determines that no efficacy has occurred (S506), and updates the lower limit value to 1.5 mg (S512). Then, the variation v(2) in an increase is 0.2 mg, and the increased intake target A(2) is 1.7 mg. 1.7 mg of the increased intake target is determined to be equal to or lower than 2.5 mg of the upper limit value (S516), and the intake target is increased to 1.7 mg.

The patient A uses the diary function of the application to input to the user terminal 120 that he or she took 1.7 mg of the smoking cessation aid A at 08:00 in the morning, and he or she woke up at 06:00 and suffered from no withdrawal symptom on the next day, May 3. In response to the side effect occurrence inquiry, the patient A inputs that a side effect (vomiting) has occurred. In the intake target determination process for the drug (S416), it is determined that a side effect has occurred (S501), the upper limit value is updated to 1.7 mg of the current intake target (S502), and the intake target is decreased (S504). The variation v(3) is 0.1 mg, and the new intake target is 1.6 mg.

It is determined that no side effect occurs (S501) and there was efficacy (S506) based on the information indicating that 1.6 mg of the smoking cessation aid A was taken at 08:00 in the morning, there was efficacy, and no side effect occurs on May 4. Then, it is determined that the new intake target in the decrease is 1.5 mg (S508), and the intake target is determined to be equal to or lower than the lower limit value since the intake target is equal to 1.5 mg of the lower limit value (S510), and 1.6 mg is maintained without changing the current target.

Individuals exhibit different side effects depending on dosage of a drug. To induce maximum efficacy, a larger amount of dosage than that of commonly determined dosage and administration may be needed. However, since it was difficult for medical practitioners to detect a patient's side effects more quickly in the medical practice in the past, a conservative dosage was established as a standard and drugs were prescribed accordingly. For this reason, some people failed to gain sufficient medical effects in administration methods in the past.

Using this example can adjust the intake target to an intake target at which no side effect occurs but efficacy occurs, that is, an effective blood concentration. When the example shown in FIG. 5 is used, the plasma drug concentration can converge to the effective blood concentration by decreasing the intake target when a side effect has occurred and increasing the intake target when efficacy is not exhibited after the side effect no longer occurs.

Since the intake target can be increased to the extent that no side effect occurs, a larger amount of drug can be taken, and a sufficient dose can be administered to each patient. Since the intake target is adjusted in a range in which no side effect occurs, an excessive burden on the patient can be avoided.

Since the intake target is decreased until it reaches the lower limit value when efficacy is exhibited, the intake target can converge on as lowest intake target as possible in the range in which efficacy is exhibited. Physical and financial burdens on the patient can be minimized.

Although the intake target adjustment process is not performed after it is determined that the intake target is not changed in this example, the process may not be performed for a predetermined period such as one week, or the process may be continuously performed every day even after it is determined that the intake target is not changed. In addition, even after it is determined to change the intake target, no change may be made for a predetermined period.

In addition, it may be determined that there is a side effect only when there are consecutive inputs indicating that there is a side effect a predetermined number of times, for example, two or more times without changing the intake target with only one user input regarding the efficacy and side effects, the intake target may be changed based on the determination result, and it may be determined that there is a side effect if a side effect occurs a predetermined number of times according to a plurality of latest histories. The presence or absence of efficacy and side effects may be stored as medical treatment history data, and the presence or absence of efficacy and side effects can be determined based on the medical treatment history data. Since the plasma drug concentration may be stable only after continuous intake for a certain period of time, a more appropriate intake target may be set by changing the intake target based on a predetermined number of user inputs or more.

Because drugs sold without instructions of a medical practitioner are administered to persons to be medicated in moderate intakes determined at the time of manufacturing as prescribed amounts, sufficient efficacy of the drugs may not be induced. The same effects can be obtained in the same mode as this example when the user or the like performs user registration in the server 130 as in the above-described modified example.

Although a patient is described as taking a drug in the same amount as the intake target in the above example, the patient may take a larger amount or smaller amount of drug than the intake target without following the intake target. For example, when medical treatment history information is received from the user terminal 120, it may be determined whether the intake target matches the actual dosage, and when they do not match, information for instructing intake according to the intake target may be transmitted to the user terminal 120, and while instruction information may be presented to the user, the intake target adjustment process 400 may be ended. Furthermore, information notifying the medical practitioner terminal 140 of the fact that the drug is not appropriately administered may be transmitted thereto.

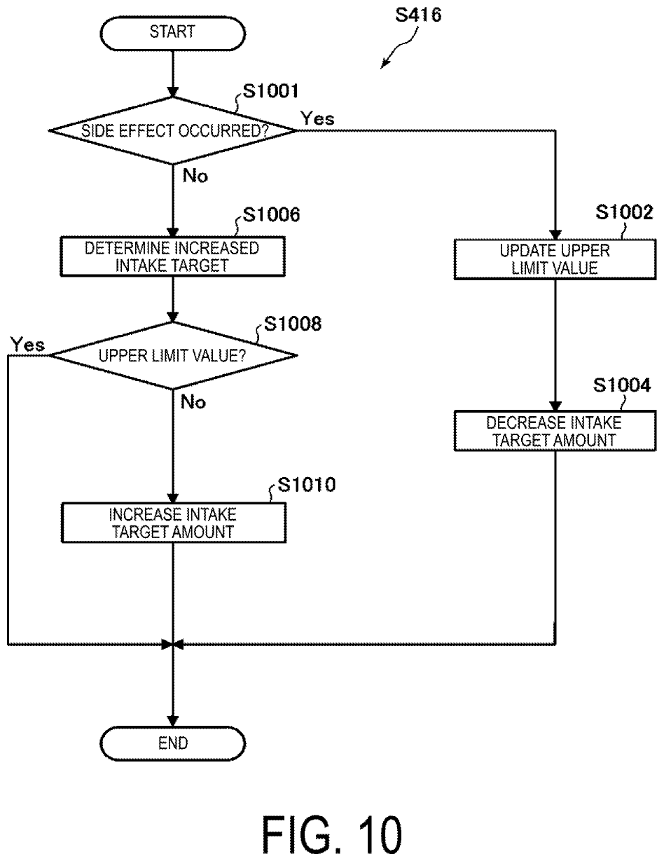

As one modified example, the intake target may be adjusted based on whether a side effect has occurred, regardless of whether the efficacy is exhibited. An example of the intake target determination process (S416) in this instance is shown in FIG. 10. First, it is determined whether a side effect has occurred based on a user response input (S1001), and if a side effect has occurred, the intake target at that time is set and updated as an upper limit value (S1002), and the intake target is decreased (S1004). When no side effect occurs, the intake target after an increase is determined (S1006), and it is determined whether the increased intake target exceeds the upper limit value (S1008). If the intake target does not exceed the upper limit value, the intake target is increased (S1010), and if the intake target exceeds the upper limit value, the intake target is not changed. Using the modified example can adjust the intake target while suppressing occurrence of side effects with a simpler configuration.

Since the intake target is increased before a side effect occurs, and the intake target is decreased after a side effect occurs until no side effect occurs so that the intake target occurs, the intake target is set as large as possible in a range in which no side effect occurs, and thus a possibility of obtaining a greater efficacy becomes higher.

Although the user terminal 120 and the server 130 are used in the above-described example as one modification, for example, the example may be implemented with the user terminal 120 having the functions of the server 130 and without using the server 130.

In the processing or operations described above, the processing or operations can be modified freely as long as there is no occurrence of contradiction in the processing or operations such as using data that is not yet supposed to be used in a corresponding step. In addition, each example described above is exemplified for our systems, devices, methods and programs, and this disclosure is not limited to the examples. Our systems, devices, methods and programs may be implemented in various forms without departing from the scope thereof.

1-17. (canceled)

18. A system that adjusts an intake target of a drug indicating an amount of the drug to be taken by a person to be medicated, the system configured to:
acquire intake information of a drug taken by a person to be medicated based on an intake target of the drug;
determine, based on the intake information, an occurrable timing at which a side effect of the drug taken is likely to occur;
present inquiry information that inquires whether a side effect has occurred in the person to be medicated at the occurrable timing;
acquire response information that is input in response to the inquiry and indicates whether a side effect has occurred;
change the intake target of the drug based on the response information; and
present the intake target changed.

19. The system according to claim 18, wherein
the occurrable timing is an occurrable period during which a side effect is likely to occur, and
presenting the inquiry information includes presenting the inquiry information in response to a presentation request of a user during the occurrable period.

20. The system according to claim 18, wherein
the occurrable timing at which the side effect is likely to occur is determined based on an occurrable timing determinant, and
the system further corrects an occurrable timing determinant based on the response information.

21. The system according to claim 20, wherein the occurrable timing determinant is an estimated time to a maximum plasma concentration.

22. The system according to claim 18, wherein
the system determines a probability of occurrence of a side effect at a timing to present inquiry information; and
presenting the inquiry is preferentially presenting a side effect in which the high probability of occurrence determined is high.

23. The system according to claim 18, wherein
the system determines a probability of occurrence of a side effect at a timing to present inquiry information; and
presenting the inquiry is presenting the probability of occurrence determined along with a side effect.

24. The system according to claim 18, wherein
the system repeatedly performs acquisition of the intake information of the drug, determination of the occurrable timing, presentation of the inquiry information, acquisition of the response information, change of the intake target of the drug, and presentation of the intake target changed, and
the change of the intake target of the drug includes increasing the intake target of the drug when the response information indicates that no side effect occurs, and decreasing the intake target of the drug when the response information indicates that a side effect has occurred.

25. The system according to claim 18, wherein
the system repeatedly performs acquisition of the intake information of the drug, determination of the occurrable timing, presentation of the inquiry information, acquisition of the response information, change of the intake target of the drug, and presentation of the intake target changed, and
the intake target of the drug is decreased after the response information indicates that a side effect has occurred, and the intake target of the drug is maintained after the response information indicates that no side effect occurs.

26. The system according to claim 18, wherein
the inquiry information includes information for inquiring whether efficacy of the drug has occurred in a person to be medicated,
the response information includes information indicating whether efficacy of the drug has occurred, and
the change of the intake target of the drug is performed based on response information including the information indicating whether the efficacy of the drug has occurred.

27. The system according to claim 26, wherein
the system repeatedly performs acquisition of the intake information of the drug, determination of the occurrable timing, presentation of the inquiry information, acquisition of the response information, change of the intake target of the drug, and presentation of the intake target changed, and
the intake target of the drug is decreased when the response information indicates that a side effect has occurred, and the intake target of the drug is increased when the response information indicates that no side effect occurs and that no efficacy of the drug occurs.

28. The system according to claim 18, wherein
the system repeatedly performs acquisition of the intake information of the drug, determination of the occurrable timing, presentation of the inquiry information, acquisition of the response information, change of the intake target of the drug, and presentation of the intake target changed, and
includes determining that the response information indicates that no side effect occurs when the response information indicates that no side effect occurs a predetermined number of times or more.

29. An electronic device that adjusts an intake target indicating an amount of a drug to be taken by a person to be medicated, the electronic device configured to:
receive intake information of a drug taken by a person to be medicated;
determine, based on the intake information, an occurrable timing at which a side effect caused by the drug taken is likely to occur;
transmit information that presents inquiry information that inquires whether a side effect has occurred in the person to be medicated at the occurrable timing;
receive response information that is input in response to the inquiry and indicates whether a side effect has occurred;
change an intake target of the drug based on the response information; and
transmit information for presenting the intake target changed.

30. A method performed by a computer that adjusts an intake target of a drug indicating an amount of the drug taken by a person to be medicated, the method comprising:
acquiring intake information of a drug taken by a person to be medicated based on an intake target of the drug;
determining, based on the intake information, an occurrable timing at which a side effect caused by the drug taken is likely to occur;
presenting inquiry information that inquires whether a side effect has occurred in the person to be medicated at the occurrable timing;
acquiring response information that is input in response to the inquiry and indicates whether a side effect has occurred;
changing the intake target of the drug based on the response information; and
presenting the intake target changed.

31. A method performed by a computer that adjusts an intake target indicating an amount of a drug to be taken by a person to be medicated, the method comprising:
transmitting intake information of a drug taken by a person to be medicated input;
presenting an inquiry based on received inquiry information that inquires whether a side effect caused by the drug taken has occurred in the person to be medicated at an occurrable timing determined based on the intake information at which the side effect is likely to occur; and
transmitting response information that is input in response to the inquiry and indicates whether the side effect has occurred.

32. A non-transitory computer readable medium storing a program that causes a computer to perform the method according to claim 30.

33. A non-transitory computer readable medium storing a program that causes a computer to perform the method according to claim 31.
